(19) United States
(12) Patent Application Publication
(10) Pub. No.: US 2010/0202932 A1
(43) Pub. Date: Aug. 12, 2010

(54) AIR MOVEMENT SYSTEM AND AIR CLEANING SYSTEM

(76) Inventor: Dennis R. Danville, Trenton, MI (US)

Correspondence Address: CARGILL & ASSOCIATES, P.L.L.C., 56 MACOMB PLACE, MT. CLEMENS, MI, 48043, US

(21) Appl. No.: 12/703,754

(22) Filed: Feb. 10, 2010

Related U.S. Application Data
(60) Provisional application No. 61/151,346, filed on Feb. 10, 2009.

(51) Int. Cl. A61L 9/00; A61L 9/20

(52) U.S. Cl. 422/121

(57) ABSTRACT

An air movement and air cleaning system which includes an air movement system preferably including fan and fan housing to prevent thermal gradients in a building or room, in combination with an air cleaning surface of at least titanium dioxide, to react with moisture in the air and an ultraviolet light source in close proximity to the air cleaning surface, such that as humidity in the air passes through the air movement system over the titanium dioxide, the ultraviolet light creates hydroxyl radicals in the presence of the titanium oxide catalytic surface thereby purifying the air that passes therethrough.

### CROSS REFERENCES TO RELATED APPLICATIONS

This application claims the benefit under 35 U.S.C. 119(e) of U.S. Provisional Application No. 61/151,346 filed on 10 Feb. 2009.

### TECHNICAL FIELD

The present invention relates generally to an air movement and air cleaning system, and more particularly it relates to an air purification device utilizing specialty coatings on components of a fan that also reduces the thermal gradient in a heated or cooled building to purify the air and normalize the temperature simultaneously.

### BACKGROUND OF THE INVENTION

Clean air is not only an occupational necessity, but it reduces costs on cleaning equipment and makes the workplace or retail operation a cleaner and healthier place to work and shop. New technologies for cleaning and moving air come out all the time, but it is imperative to provide a simple, inexpensive, yet effective system to make clean air a true reality in most environments. Conventional air movement and/or destratification systems have traditionally been utilized to help circulate air to equalize temperatures within large areas. Industrial plants having high ceilings, as well as big box stores, attempt to maintain uniform temperatures throughout the vertical airspace. Since this is not traditionally an easy thing to do, it appears to be a problem. Without using air ducts for flowing air from top to bottom, one prior art method utilized various fans to circulate the air. In another prior art attempt to solve this issue, heating distributors were used to blow warm air from the ceiling down towards the floor of the building in order to prevent temperature gradience.

Some prior art methods have used ultraviolet radiation to sterilize the air from the ceiling as it passes it back down towards the floor. Other systems have used photocatalytic oxidation in order to provide cleaner air, but those systems have not met with much financial success because they are too bulky, or they are providing sprays, or other undesirable disinfecting side effects that proved to be untenable for commercial use.

Consequently, HVAC contractors have identified a need for providing an air distribution system that will both redistribute heated air without a thermal gradient throughout the vertical in buildings, as well as producing clean air solutions for residential, commercial, and industrial applications.

Smoke abatement applications would be most advantageous in really smokey environments, such as in shops that generate welding smoke. In such an environment, an air cleaning system is just as desirable as a system that can redistribute heated air. Providing a combination device would be highly desirable. Since smoke contains environmental contaminants and pollutants, sometimes including carbon monoxide, nitrous oxide, sulfur-containing compositions, and other undesirable pollutants which, when inhaled by welding operators, causes health problems over an extended period of exposure, cleaning the smoke out of the air the welding operators breathe would be a great help. The same would be true for other contaminants such as VOC's, odors, bacteria, germs, fungus, etc. in many work, health care and living environments.

In other contaminated work areas, various noxious fumes and smokes are generated by machines or tanks within the industrial environment, and the air needs to be cleaned as it is distributed, or else the air must be sent to a scrubber prior to sending it to an exhaust vent. The ability to break down the components of the polluted gas will generate commercial success.

There are many ways, conventionally, for cleaning the air, although not many of them come in contact with all of the air being distributed within a room. For example, the air can be ducted through a duct to an air purification system, such as a scrubber, and then reintroduced back into the room. For open air cleaning, such as bars and restaurants and beauty salons, where cigarette smoke and ammonia containing gasses are given off, the “smoke burners” have a difficult time keeping up with the volume entirely. When such a system is utilized in an even larger area, the problems with these systems rise exponentially. When applied to a big box store or an industrial building with 20 to 30 foot high ceilings, scrubbing the air without duct-work is nearly impossible.

Therefore, it would be an advantage to the industry to be able to provide a device which would be able to both purify the air and redistribute or destratisfy the air to alleviate thermal gradiency in a single device. It would further be an advantage to be able to purify the air without any consumable filtration system. In addition, a system that would also be self-cleaning in the process, would be extremely beneficial.

### SUMMARY OF THE INVENTION

Accordingly, there are many aspects of the present invention, wherein a first aspect includes an air movement system in combination with an air purification system. The Air purification aspect includes the provision of a catalytic surface, and in one aspect, applying it directly onto the fan blades of an air movement system box fan, in order to react with and render inert various organic and inorganic compounds present in the environment of the building or room. The catalytic surface may utilize titanium dioxide for a base catalyst as a coating on at least the fan blades of an air movement system for purifying the air while also thermally stabilizing the temperature in a room. The air movement system may be installed anywhere, including near the ceiling while utilizing its catalytic surface in conjunction with an ultraviolet light structurally situated within the air movement system such that the titanium dioxide catalyst purifies the air passing therethrough.

This air purification is achieved through the production of hydroxyl radicals, which are among the strongest oxidizing species. These hydroxyl radicals detach double carbon bonds, thereby breaking down the constituents into single carbon bond intermediates which are then reduced into carbon dioxide and water. These particles act as very powerful disinfecting agents by oxidizing the cellular structure of microorganisms, causing rupture and leakage of the molecule's vital composition. Therefore, this causes the decomposition of the pollutant.

When bacteria, germs, fungus, odors and VOC's are in the presence of titanium dioxide and ultraviolet light, a photocatalytic reaction occurs, yielding deodorization, air purification, sterilization, and self cleaning of the unit. The photocatalytic surface of the present invention may include a base catalyst of titanium dioxide, and may also be combined with a mixture of oxides including oxides and/or mixed oxides of the transition metals chromium, manganese, cobalt, nickel, copper, vanadium, zinc, molybdenum and tungsten. Of special interest is the anatase crystal of titanium oxide because it creates an energy band gap that turns the energized crystal into an electron magnet. When water molecules present is steam in the air, pass over the energized titanium dioxide, their electrons get pulled off the water molecule, creating two hydroxyl ions, or as they are known, hydroxyl radicals. These hydroxyl radicals generated during the photocatalytic process tend to break up any and all organic molecules that pass therethrough. Organic molecules may include bacteria, germs, oils, and VOC's. These reactions take so fast that it is impossible to physically view the reaction, and they continue to happen as long as there is ultraviolet light, water vapor, and titanium oxide present.

Titanium oxide should comprise at least about twenty to fifty percent by weight of the catalyst and may comprise up to one hundred percent by weight of the catalytic coating. The binder or solvent is not included in the weight of the catalytic coating. The other oxides or mixed oxides of the transition metals described herein above may comprise the balance of the weight of the coating on the photocatalytic surface, or it may be a surface effect on a solid titanium oxide piece. Of special interest may be titanium dioxide of the anatase crystal form, which would have a relatively high specific surface area of at least one hundred meters squared per gram, which is commercially available. Not only that, but at room temperatures where this system is expected to be used, the anatase crystal form is quite thermally stable, and will not transit into the rutile form. The commercially available titanium dioxide may be milled to a fine mesh size and mixed with a binder in order to be applied to the fan blades of the air movement system. This catalyst is especially useful for the elimination of bacteria, germs, mold, and VOC's.

This photocatalytic coating on the fan blades of the air movement system provide several advantages, i.e. the system itself cleaning and provides a biofilm barrier along with odor abatement and smoke control. The self cleaning feature means that windows, building surfaces, solar panels, antimicrobial infection control systems, and mold remediation is automatic without any maintenance needed. Although a thin film of the catalyst is highly effective and may be utilized for the system, it is also within the scope of the invention for the fan blades to be formed out of the titanium dioxide and balance of other materials by itself.

The present invention, therefore, discloses a unique combination of an air movement circulatory system which creates a vortex by the movement of its fan blade as well as organizing the movement of the air with the purification properties of an ultraviolet light and a catalytic cleaning process of a titanium dioxide coating or bulk through effect.

The air movement circulation system creates an air envelope which has air continuously passing thereover the surfaces. This new process continuously cleans and purifies the air as it passes over the coated surface of the fan blades while it also normalizes the thermal gradient in a room in which the air movement system is mounted.

An additional aspect of the present invention is that rusting of the components within the building are retarded as a result of the continuous and organized air circulation path which may lower the dew point by not allowing water in the air, usually present as normal humidity, to stagnate on any rustable surface.

In yet another aspect of the present invention, it may be noted that since the air passing through the air envelope has been electronically charged by the air purification process, acceleration of the purification results, as well as further retardation of the rusting of metallic components in the room.

In yet another aspect of the present invention, all of the fan components may be coated or made from the catalytic material described hereinabove. The various components may include not only the fan blades, but the housing, the vent housings, the respective side walls, the baffle, and any other components of the air movement system or plenum which may ever come in contact with the air during the down draft operation.

Although the invention will be described by way of examples hereinbelow for specific embodiments having certain features, it must also be realized that minor modifications that do not require undo experimentation on the part of the practitioner are covered within the scope and breadth of this invention. Additional advantages and other novel features of the present invention will be set forth in the description that follows and in particular will be apparent to those skilled in the art upon examination or may be learned within the practice of the invention. Therefore, the invention is capable of many other different embodiments and its details are capable of modifications of various aspects which will be obvious to those of ordinary skill in the art all without departing from the spirit of the present invention. Accordingly, the rest of the description will be regarded as illustrative rather than restrictive.

### BRIEF DESCRIPTION OF THE DRAWINGS

For a further understanding of the nature and advantages of the expected scope and various embodiments of the present invention, reference shall be made to the following detailed description of the invention, and when taken in conjunction with the accompanying drawings, in which like parts are given the same reference numerals, and wherein:

FIG. 1A is a perspective environmental view of an air movement and air cleaning system made in accordance with the present invention;

FIG. 1B is a perspective environmental view of another aspect of the present invention wherein the air movement and air cleaning system is hung lower from a ceiling;

FIG. 2 illustrates a detail of the fan housing of one aspect of the present invention; and

FIG. 3 a top plan view of the air movement and air cleaning system.

### DETAILED DESCRIPTION OF THE INVENTION

In accordance with the present invention, there is provided a new and novel device which can both clean air as well as redistribute and destratify it to alleviate thermal gradiency in a large volume industrial or commercial building setting or any area where destratification or air quality presents a problem. The device of the present invention includes a coating of titanium dioxide, or TiO₂, on at least most of the downdraft fan. The fan will have a conical shaped plenum, which may be mounted in the surface of the ceiling, or may be suspended a sufficient length down from the ceiling in order to allow for air mixing above the fan, whereby the effective heated area in the building actually saves heating the top ten or twenty feet of the building. The advantage of the suspended downdraft fan is that it effectively lowers the ceiling due to the open-air/open-back mounting.

In order to address cleaning such a large volume of air, a titanium dioxide photocatalytic oxidation coating has been placed on the blades and at least most, if not all, of the surface areas of the destratification/cleaning unit, and when combined with an ultraviolet light, the blade and any other coated surface of the destratification/cleaning system becomes reactive for commercial odors and bacteria elimination along with VCO's, germs and fungus. By itself, TiO₂ is an inert coating. When ultraviolet light strikes the TiO₂ coated surface, in the presence of water vapor, such as humidity in the air, the TiO₂ creates hydroxyl radical ions which are very powerful cleaning agents. The combination of ultraviolet disinfecting activity with the cleaning benefit of TiO₂ provides industry with a very powerful air movement system to keep indoor air spaces clean and bacteria free, while redistributing heated air to eliminate heating gradiencies.

Hydroxyl radical ions are inherently utilized by nature as a cleansing agent in the earth's atmosphere. A wide variety of pollutants, including smoke, carbon monoxide, methane, and nitrogen are purged from the atmosphere when contacted with hydroxyl radicals. Although hydroxyl radicals have an extremely brief lifetime, they are among some of the strongest oxidizing species when reacting with pollutants. These hydroxyl radical ions have been tested to be even stronger than chlorine, ozone, or peroxide as they detach double carbon-carbon bonds to yield a single carbon bond intermediary from carbonaceous constituents, and then the single carbon bond intermediary breaks down into carbon dioxide and water.

Decomposition of the pollutants results from oxidizing micro-organism cell wall structures, whereby rupture and leakage of the cell walls are affected by these very powerful disinfecting agents. In essence, the TiO₂photocatalytic reaction exhibits deodorization, self-cleaning, air purification, and significant sterilization of VOC's, odors, bacteria, germs, and fungus. The TiO₂ coating includes anatase crystals of TiO₂, and when light in the UV-A frequency range hits such an anatase crystal; an energy band gap is created that turns the anatase crystal into a magnet for electrons. The electrons are generated when water molecules (i.e. steam or humidity in the air) come into contact with the TiO₂ crystals, pulling the electrons off the water molecules. Consequently, water molecules are turned into two hydroxyl ions, and generating one of the most powerful scrubbing agents in the world. The hydroxyl radical ions have a 400% greater oxidative power than chlorine. Therefore, its ability to break up organic molecules is significantly greater than conventional methods of air cleansing.

Many pollutants in the air incorporate organic molecules, whether they are smoke, oils, VOC's, or parts of cellular walls of microbes. The photocatalytic reaction available through the combination of ultraviolet light and TiO₂ can actively attack and break up any of these organic molecules, and will continue to happen as long as contact with air containing water vapor, exposure to ultraviolet light, and exposure to the TiO₂ coating continues to occur.

The real advantages of a photocatalytic degradation system is that the pollutants are really destroyed rather than collected on a substrate, all while at ambient temperatures and pressures.

The photocatalytic reaction is effective on a large range of pollutants and is economical and easy to maintain because there aren't any filters that need to be replaced nor are there any consumables to create the effect. In essence, once one of these destratification/air cleaning systems is installed, it can be mounted and forgotten.

Furthermore, a large advantage to the present invention is that the photocatalytic reaction means that there is no dust build-up on the blades of the downdraft fan or other areas that have been coated, rendering these units to be nearly maintenance-free. Mold and mildew are alleviated to a large extent, because the photocatalytically active component renders mold and mildew inactive, so maintenance issues relating to this problem are diminished.

An air movement and air cleaning system in accordance with the present invention may basically include a housing adapted for placement in a building for air flow communication within the housing with a means for drawing air from the building through the housing, where the means for drawing air is close to or inside the housing and wherein air flow communicates thereby.

An ultraviolet light means is attached within said housing that radiates ultraviolet light, and a surface of a photocatalytically active material also within said housing may be illuminated by and is within target range of said ultraviolet light radiation, such that the ultraviolet light radiation impinges on at least a portion of the surface of a photocatalytically active material. The air flowing through said housing is drawn therethrough and contacts the surface of photocatalytically active material in the presence of the ultraviolet light radiation means, so that air purification may be achieved.

In another aspect of the invention, the housing is adapted for suspension from a ceiling to provide downdrafting of the air flow to alleviate thermal gradients in the building. Further, the housing that contains the means for drawing air from the building may include a fan blade configuration. In addition to the fan blades being coated, the housing may be coated with the photocatalytically active material so that air contacting the housing as it passes therethrough is activated. Both the housing and the fan blade that is coated with the photocatalytically active material are positioned so that air contacting the housing as it passes therethrough is activated. The entire air movement system is optionally coated with the photocatalytically active material so that air contacting the system as it passes therethrough is activated.

The ultraviolet light means utilized by the present invention may be selected from the group consisting of high intensity UV, low intensity UV, broad spectrum UV, germicidal UV, 254 NM UV, advanced oxidation UV, multiple oxidizer UV and combinations thereof. Although the inventor has experimented with many different types of UV light, the broad spectrum UV appears to have the most applications for air purification. Regardless, all possible UV applications are within the scope of this invention. For most of the catalytic surfaces described herein, the inventor has found that an ultraviolet light source emitting wavelengths of light that are shorter than 387 nanometers will activate the energy of any catalyst used. References state that 387 NM is the activation energy of pure titanium dioxide in the anatase crystal form, and shorter wavelengths will activate any of the other combinations detailed herein.

The surface of a photocatalytically active material may include materials selected from the group consisting of titanium oxide, and oxides, mixed oxides, nitrides and chromites of transition metals selected from the group consisting of chromium, manganese, cobalt, nickel, copper, vanadium, zinc, molybdenum and tungsten. These additional components possess varying properties that may be desirable.

For the purpose of yet another aspect of the present invention, and at the temperatures that it normally encounters, the photocatalytically active material includes the anatase crystal of titanium oxide, because it will not convert to the rutile crystal at room temperatures.

The application of the photocatalytically active coating on the various surfaces such as the fan blades, the housing, or any other air movement system component of at least a portion of titanium oxide may be made by spraying a slurry of titanium oxide in water onto the surface as a coating. Further, a solid piece of titanium oxide may be formed into those components. Increasing the surface area may be beneficial to promote further contact with the air that contains humidity, bacteria, mold, fungus, etc. In that regard, another aspect of the invention calls for a corrugated surface to increase surface area where the air flows over. Further, the invention may comprise the addition of a highly porous high surface component near the ultraviolet light means for the air to flow through. Increasing the surface area, no matter how it may be effected for a particular purpose, is within the scope of my invention.

The surface of the photocatalytically active material can be coated onto a substrate selected from the group consisting of coated fan blades, solid titanium dioxide honeycomb structure, titanium oxide coated expanded metal, coated housing components, partially coated and partially solid titanium oxide components.

In another aspect of the present invention for an air movement and air cleaning system, a housing coated at least partially with a titanium oxide-containing coating is included. The housing may be adapted for placement in a building for air flow communication within the housing. Since the building must have some humidity, the photocatalytic reaction can take place when the air containing the humidity comes within radiation distance of the UV light. If the fan blades are coated, at least partially, and the UV light can impinge on the coating, then it is likely that reaction will take place where hydroxyl radicals are produced. As described in more detail hereinabove, the radicals act as disinfectants, destroying any mold, mildew, bacteria, etc. in the air passing thereover. presence of the ultraviolet light radiation means.

Of special interest is that the housing may be adapted for suspension from a ceiling to provide downdrafting of the air flow past the photocatalytically active material to purify the air and also to alleviate thermal gradients in the building. It is an additional aspect of the present invention for the present invention to be mounted on a wall, or to be provided as a portable unit that can be moved from room to room within a building. Although these embodiments are not specifically shown in drawings, it is for one of ordinary skill in the art to know how to mount a fan in a housing sideways on a wall, or to place it in a portable unit.

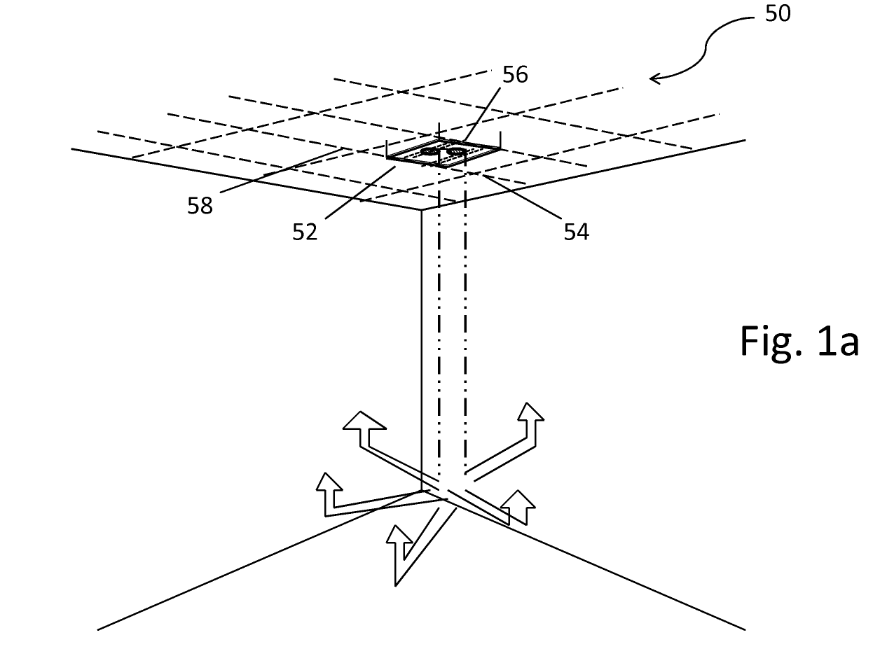

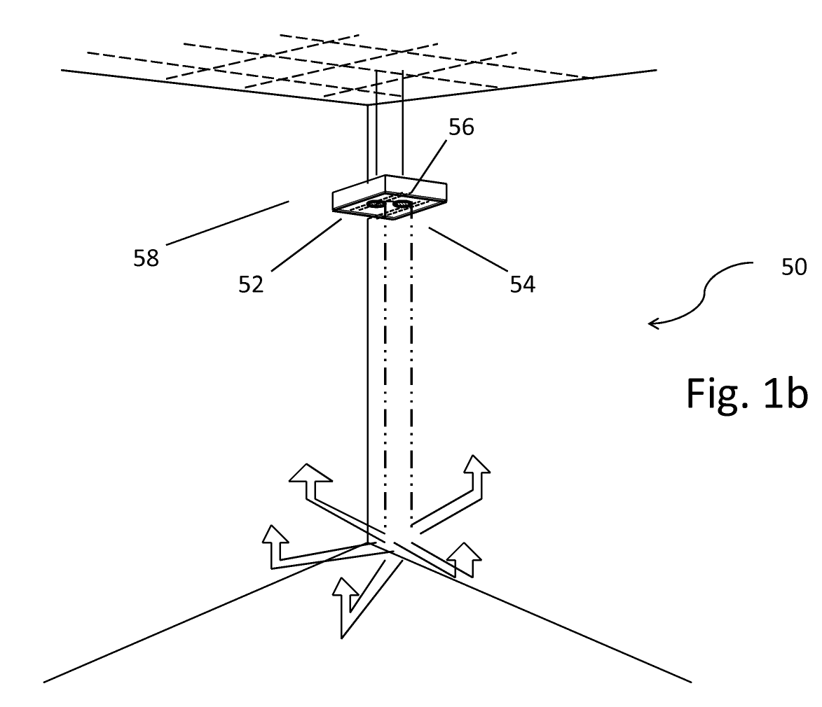

Looking first to FIG. 1A, the numeral 50 generally denotes an air-purification and distribution downdraft device made in accordance with the present invention. Assembly 52 is a suspended ceiling frame structure including ledges 54 of the cross braces. A center leg 56 is suspended from a plurality 58 of cross braces. Air circulates through the downdraft fan 50 to cause a vertical redistribution of warm air from the ceiling to the floor. By redistributing the heated air from the ceiling to the floor, a temperature gradient of less than 2° or 3° F. is easily achieved.

FIG. 1B illustrates a suspended downdraft fan to allow up to twenty feet downward mounting position from the ceiling in order to effectively reduce the volume of air needed to be heated. In essence, the downdraft of air hits the floor and by the low pressure center created by the destratification unit and the resulting organized air flow, is drawn back by fan 56 as it reaches the top of the ceiling in the room or the level of the mounted unit in an open air application. The downdraft redistributes the air from the vertical height of the fan rather than from directly underneath the ceiling. Consequently, the heated air stays within the “air envelope' created by the circulation process and does not rise all the way to the ceiling/roof (in the open air application) before it has to be returned.

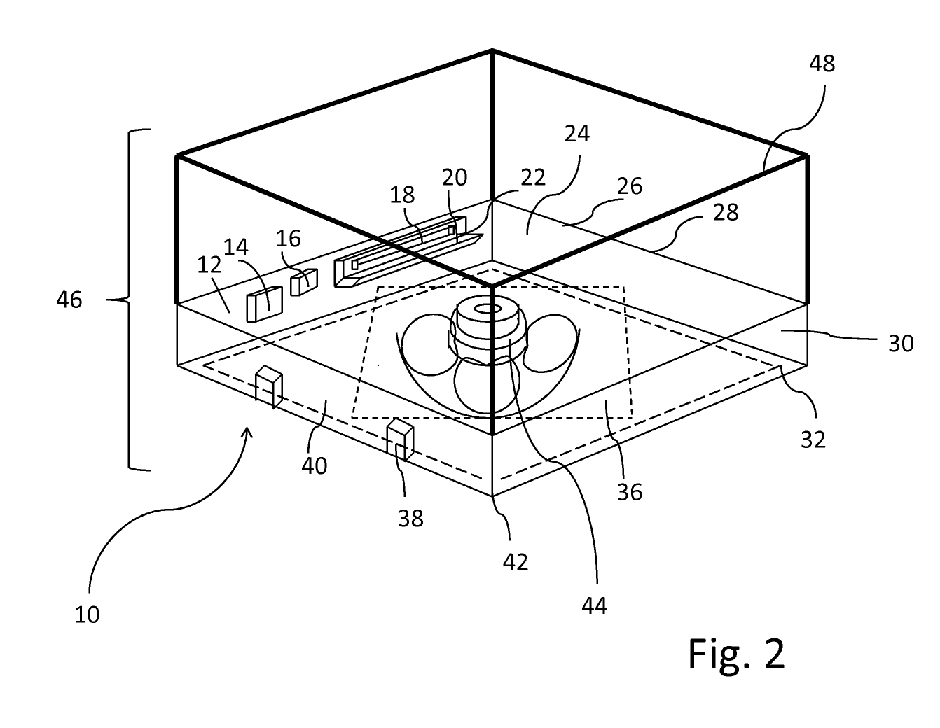

FIG. 2 is the downdraft fan device of the present invention, and is generally denoted by numeral 10 and includes a TiO₂ coated side wall 12. A rheostat 14 for controlling the speed of the TiO₂ coated fan 10 is mounted to side wall 12. A switch 16 is mounted on side wall 12 for energizing a TiO₂ coated ultraviolet light 18. Ultraviolet light 18 includes a horizontally extending portion 20 and an L-shaped shield 22 is attached thereto in order to prevent UV radiation from passing downwardly into the room area below. A plenum cover 24 also may include a TiO₂ coating, as well as a TiO₂ coated housing 26. A plurality of side walls, respectively 28 and 30 may further include a TiO₂ coating. The invention may also advantageously incorporate an inwardly extending lip 32 which is normal to the respective side walls, and may also be TiO₂ coated, as well as baffle 36.

Still looking at FIG. 2, a baffle 36 may be TiO₂ coated, and will help to direct the air in a downwardly extending direction for redistribution of heated air throughout the vertical of the building. A grill (not shown) is mounted with mounting clip 38 to cover TiO₂ coated fan 44. The plenum 48 may be any height, but may be preferably 6-36 inches tall. The TiO₂ coating on all the components shown in FIG. 2 will help to provide the photocatalytic reaction desired while moving the air.

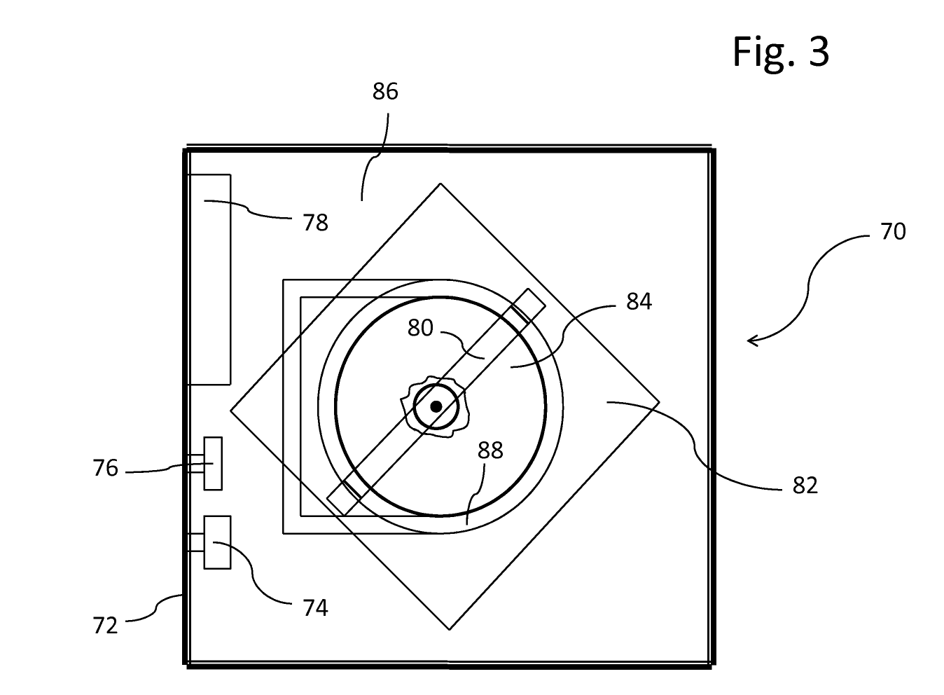

Looking lastly at FIG. 3, a top plan view of the air circulating and air purifying fan made in accordance with the present invention is shown and generally denoted by numeral 70. Air circulating and purifying fan 70 includes TiO₂ coated side walls 72 and an ultraviolet light 78 for activating the TiO₂ coating on fan blade 80 in the presence of normal room humidity. A rheostat 74 may control the speed of the fan and switch 76 energizes the ultraviolet light 78. As air is recirculated by the fan 80 through its orifice 84, the purified heated air is plunged downward towards the floor and the air is replenished through inlet passage 86 or drawn from above the unit in a open air application. Even the fan flange 88 can be coated with TiO₂ to yield more photocatalytic surface area for contacting pollutants in the air being urged downwardly to maximize the air-cleaning possibilities of the present invention. This drawing show a square containing unit that may be particularly suited for suspended ceiling applications, the invention also works and can be used in different shaped containers such a round or circular designs. In addition, the system may be used with or without an ultraviolet light depending upon the existing lighting in the area application and if ultraviolet light sources are already present. The system also may be used in conjunction with a filter attached or inserted in the unit to filter other contaminants, i.e. large particulates, etc., as may be needed for a particular application.

The foregoing description of a preferred embodiment of the invention has been presented for purposes of illustration and description. It is not intended to be exhaustive or to limit the invention to the precise form disclosed. Obvious modifications or variations are possible in light of the above teachings with regards to the specific embodiments. The embodiment was chosen and described in order to best illustrate the principles of the invention and its practical applications to thereby enable one of ordinary skill in the art to best utilize the invention in various embodiments and with various modifications as are suited to the particular use contemplated.

What is claimed is:

1. An air movement and air cleaning system, comprising:
a housing adapted for placement in a building for air flow communication within the housing;
a means for drawing air from the building through the housing, said means for drawing air being proximate to the housing and wherein air flow communicates thereby;
an ultraviolet light means attached within said housing that radiates ultraviolet light; and
a surface of a photocatalytically active material also within said housing, and said surface of photocatalytically active material being within target range of said ultraviolet light radiation, such that the ultraviolet light radiation impinges on at least a portion of the surface of a photocatalytically active material;
whereby air flowing through said housing is drawn therethrough and contacts the surface of photocatalytically active material in the presence of the ultraviolet light radiation means.

2. The system of claim 1, wherein the housing is adapted for suspension from a ceiling to provide downdrafting of the air flow to alleviate thermal gradients in the building.

3. The system of claim 1, wherein the housing contains the means for drawing air from the building, and wherein the means for drawing air is a fan blade configuration.

4. The system of claim 1, wherein the housing is coated with the photocatalytically active material so that air contacting the housing as it passes therethrough is activated.

5. The system of claim 1, wherein both the housing and the fan blade is coated with the photocatalytically active material so that air contacting the housing as it passes therethrough is activated.

6. The system of claim 1, wherein the entire air movement system is coated with the photocatalytically active material so that air contacting the system as it passes therethrough is activated.

7. The system of claim 1, wherein the ultraviolet light means is selected from the group consisting of high intensity UV, low intensity UV, broad spectrum UV, germicidal UV, 254 NM UV, advanced oxidation UV, multiple oxidizer UV and combinations thereof.

8. The system of claim 1, wherein the surface of a photocatalytically active material includes materials selected from the group consisting of titanium oxide, and oxides, mixed oxides, nitrides and chromites of transition metals selected from the group consisting of chromium, manganese, cobalt, nickel, copper, vanadium, zinc, molybdenum and tungsten.

9. The system of claim 1, wherein the surface of a photocatalytically active material includes the anatase crystal of titanium oxide.

10. The system of claim 1, wherein the surface of a photocatalytically active material includes the application of at least a portion of titanium oxide onto the surface of a high surface area housing component, such as a corrugated surface to increase surface area where the air flows over.

11. The system of claim 1, wherein the surface of a photocatalytically active material further comprises the addition of a highly porous high surface component near the ultraviolet light means for the air to flow through.

12. The system of claim 1, wherein the surface of a photocatalytically active material includes a substrate selected from the group consisting of coated fan blades, solid titanium dioxide honeycomb structure, titanium oxide coated expanded metal, coated housing components, partially coated and partially solid titanium oxide components.

13. An air movement and air cleaning system, comprising:
a housing coated at least partially with a titanium oxide-containing coating, said housing being adapted for placement in a building for air flow communication within the housing;
a fan for drawing air from within the building through the housing, said fan for drawing air being proximate to the housing and wherein air flow communicates thereby;
an ultraviolet light means attached within said housing that radiates ultraviolet light; and
fan blades being at least partially coated with a photocatalytically active material also within said housing, and said photocatalytically active material being within proximate target range of said ultraviolet light radiation, such that the ultraviolet light radiation impinges on at least a portion of the surface of the photocatalytically active material;
whereby air flowing through said housing is drawn therethrough and contacts the surface of the photocatalytically active material in the presence of the ultraviolet light radiation means.

14. The system of claim 13, wherein the housing is adapted for suspension from a ceiling to provide downdrafting of the air flow past the photocatalytically active material to purify the air and also to alleviate thermal gradients in the building.
